(19) United States
(12) Patent Application Publication
(10) Pub. No.: US 2013/0345751 A1
(43) Pub. Date: Dec. 26, 2013

(54) SUTURE ANCHOR INSERTION APPARATUS, SUTURE ANCHOR, AND METHODS OF USING THE SAME

(71) Applicant: TORNIER, INC., Bloomington, MN (US)

(72) Inventor: Clinton BECK, Fort Wayne, IN (US)

(21) Appl. No.: 13/923,114

(22) Filed: Jun. 20, 2013

Related U.S. Application Data
(60) Provisional application No. 61/662,294, filed on Jun. 20, 2012.

(51) Int. Cl. A61B 17/04

(52) U.S. Cl. CPC A61B 17/0401
USPC 606/232

(57) ABSTRACT

A suture anchor insertion apparatus includes an insertion device having an insertion sheath. The insertion sheath includes an inner chamber and a distal end. The distal end is adapted to penetrate bone of a subject, and the distal end defines an opening in communication with the inner chamber. An anchoring device is carried by the insertion sheath within the inner chamber. The anchoring device includes a distal surface adapted to engage the bone of the subject when the distal end of the insertion sheath penetrates the bone of the subject. The anchoring device is adapted to pass through the opening to detach from the insertion sheath and couple to the bone of the subject. A suture is coupled to the anchoring device and is adapted to couple to soft tissue of the subject.

### CROSS-REFERENCE TO RELATED APPLICATION

This application claims the benefit of U.S. Provisional Patent Application Ser. No. 61/662,294, filed on Jun. 20, 2012 and entitled “DIRECT INSERTION ANCHOR SYSTEM”, which is hereby incorporated by reference in its entirety.

### TECHNICAL FIELD

The present invention relates to insertion devices for suture anchors for coupling soft tissue of a subject to bone of the subject. In particular, the present invention relates to insertion devices for toggling suture anchors and methods of using the same without need for pre-drilling, tapping, or punching the bone of the subject.

### BACKGROUND

In some situations, biological soft tissue, such as ligaments, tendons, and/or cartilage, becomes separated from associated bone. Surgical procedures are typically used to reattach the soft tissue to the associated bone. Various devices, including sutures, screws, staples, wedges, and plugs have been used to secure soft tissue to bone. In addition, structures referred to as suture anchors have been developed to reattach soft tissue to associated bone.

Some suture anchors are inserted into pre-drilled bone holes. Other suture anchors are referred to as “direct insertion” anchors because they do not need pre-drilled bone holes. For example, some direct insertion suture anchors are threaded to facilitate self-tapping into bone, and other direct insertion suture anchors have a “spike” shape to facilitate self-punching into bone. As such, direct insertion suture anchors typically require less operating time for insertion compared to anchors that require pre-drilled bone holes.

Direct insertion anchors typically comprise relatively hard materials, such as metals, to facilitate, for example, self-tapping or self-punching into relatively hard cortical bone. As such, direct insertion anchors do not typically include other materials used in biomedical applications, such as soft polymer materials preferred by some surgeons.

### SUMMARY

In some embodiments, a suture anchor insertion apparatus includes an insertion device having an insertion sheath. The insertion sheath includes an inner chamber and a distal end. The distal end is adapted to penetrate bone of a subject, and the distal end defines an opening in communication with the inner chamber. An anchoring device is carried by the insertion sheath within the inner chamber. The anchoring device includes a distal surface adapted to engage the bone of the subject when the distal end of the insertion sheath penetrates the bone of the subject. The anchoring device is adapted to pass through the opening to detach from the insertion sheath and couple to the bone of the subject. A suture is coupled to the anchoring device and is adapted to couple to soft tissue of the subject.

In some embodiments, a suture anchor insertion apparatus includes an insertion device having an insertion sheath. The insertion sheath defines a longitudinal axis of the insertion device. The insertion sheath includes an inner chamber and a distal end. The distal end is adapted to penetrate bone of a subject, and the distal end includes a distal edge disposed in a distal plane. The distal plane is disposed at an obtuse angle relative to the longitudinal axis. An opening is defined by the distal edge and is disposed in the distal plane, and the opening is in communication with the inner chamber. An anchoring device is carried by the insertion sheath within the inner chamber. The anchoring device includes a distal surface that is substantially disposed in the distal plane. The anchoring device is adapted to pass through the opening to detach from the insertion sheath and couple to the bone of the subject. A suture is coupled to the anchoring device and is adapted to couple to soft tissue of the subject.

In some embodiments, a suture anchor insertion apparatus includes an insertion device having an insertion sheath. The insertion sheath includes an inner chamber and a distal end. The distal end is adapted to penetrate bone of a subject, and the distal end defines an opening in communication with the inner chamber. The opening has a shape. An anchoring device is carried by the insertion sheath within the inner chamber. The anchoring device includes a distal surface. The distal surface is disposed adjacent the opening and substantially includes the shape of the opening. The anchoring device is adapted to pass through the opening to detach from the insertion sheath and couple to the bone of the subject. A suture is coupled to the anchoring device and is adapted to couple to soft tissue of the subject.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a perspective view of an exemplary suture anchor insertion apparatus according to embodiments of the present invention.

FIG. 2 illustrates a partial perspective view of the suture anchor insertion apparatus of FIG. 1. An insertion sheath is partially transparent and a suture is hidden to illustrate other components disposed within the insertion sheath.

FIG. 3 illustrates another partial perspective view of the suture anchor insertion apparatus of FIG. 1. The insertion sheath is partially transparent to illustrate components disposed within the insertion sheath.

FIG. 4 illustrates a partial side view of the insertion sheath of the suture anchor insertion apparatus of FIG. 1.

FIG. 5 illustrates another partial side view of the insertion sheath of the suture anchor insertion apparatus of FIG. 1.

FIG. 6 illustrates a front view of the insertion sheath of the suture anchor insertion apparatus of FIG. 1.

FIG. 7 illustrates a perspective view of an anchoring device of the suture anchor insertion apparatus of FIG. 1.

FIG. 8 illustrates a side view of the anchoring device of the suture anchor insertion apparatus of FIG. 1.

FIG. 9 illustrates a perspective view of an exemplary anchoring device of a suture anchor insertion apparatus according to embodiments of the present invention.

FIG. 10 illustrates a perspective view of an exemplary anchoring device of a suture anchor insertion apparatus according to embodiments of the present invention.

FIG. 11 illustrates a perspective view of an exemplary anchoring device of a suture anchor insertion apparatus according to embodiments of the present invention.

FIG. 12 illustrates a partial cross sectional side view of an exemplary set of anchoring devices of a suture anchor insertion apparatus according to embodiments of the present invention.

FIG. 13 illustrates a side view of an exemplary anchoring device of a suture anchor insertion apparatus according to embodiments of the present invention.

FIG. 14 illustrates a partial cross sectional side view of an exemplary anchoring device of a suture anchor insertion apparatus according to embodiments of the present invention.

FIG. 15 illustrates an exemplary method for coupling soft tissue of a subject to bone of the subject according to embodiments of the present invention.

FIG. 16 illustrates positioning a suture anchor insertion apparatus proximate to bone of the subject during the method of FIG. 15.

FIG. 17 illustrates the suture anchor insertion apparatus penetrating the bone of the subject during the method of FIG. 15.

FIG. 18 illustrates deploying an anchoring device from an insertion sheath of the suture anchor insertion apparatus and into the bone of the subject during the method of FIG. 15.

FIG. 19 illustrates removing the insertion sheath from the bone of the subject during the method of FIG. 15.

FIG. 20 illustrates applying tension to a suture coupled to the anchoring device to pivot the anchoring device within the bone of the subject during the method of FIG. 15.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

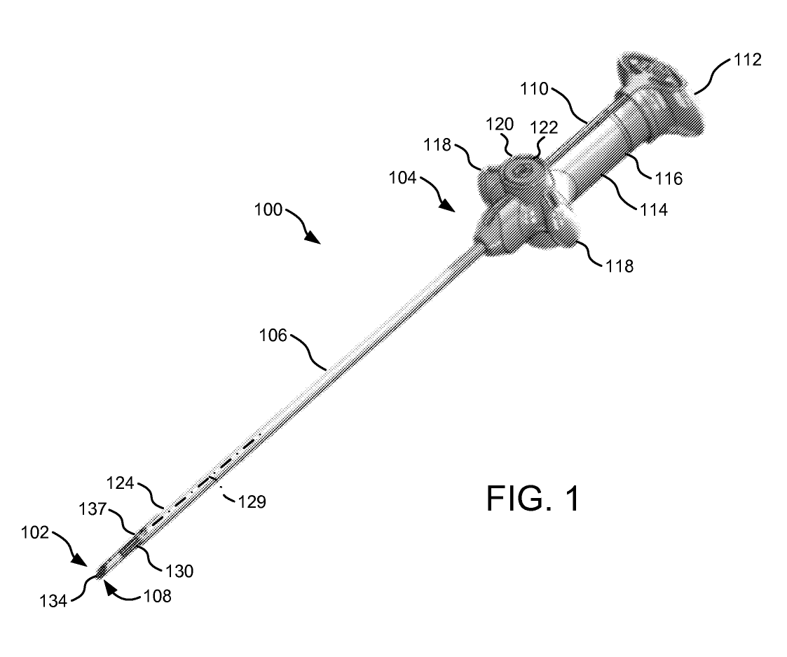

FIGS. 1-3 illustrate an exemplary suture anchor insertion apparatus 100 according to embodiments of the present invention. The apparatus 100 deploys a suture anchoring device, or anchor, 102 into bone of a patient or subject such that the anchoring device 102 may couple soft tissue, such as ligaments, tendons, cartilage, combinations thereof, and the like, to the bone of the subject. The apparatus 100 may deploy the anchoring device 102 without previously drilling, tapping, or punching the bone to form an anchor-receiving hole (by using the apparatus 100 itself or any other tools).

The apparatus 100 generally includes an insertion device or inserter 104 that is manipulated by a user (for example, a surgeon). The insertion device 104 includes an elongated insertion sheath 106. The insertion sheath 106 includes an open distal end 108 that is adapted to penetrate the bone of the subject. The insertion sheath 106 also detachably carries the anchoring device 102 in the distal end 108. The anchoring device 102 engages the bone of the subject when the insertion sheath penetrates the bone of the subject. Additionally, the insertion sheath 106 supports and reinforces the anchoring device 102 when the insertion sheath 106 penetrates the bone of the subject. As such, the anchoring device 102 may be formed by a variety of materials, including relatively hard materials (such as metals) and relatively soft materials. Relatively soft materials include materials preferred by some surgeons (for example, polyether ether ketone (PEEK)), bioabsorbable materials (for example, polymer-based bioabsorbable materials or magnesium-based bioabsorbable materials), and the like. These aspects and the above components are described in further detail below.

Referring particularly to FIG. 1, the insertion device 104 includes a handle 110 that may be grasped and manipulated by a user to position the apparatus 100 relative to the bone of the subject. The handle 110 may be formed of various appropriate materials, such as metals, plastics, combinations thereof, and the like.

In some embodiments, the handle 110 includes a proximal handle 112. The proximal handle 112 may be impacted or struck by a tool, such as a mallet (not shown) to cause the insertion sheath 106 to penetrate the bone of the subject. The proximal handle 112 may also include a slot (not shown) for detachably securing a suture coupled to the anchoring device 102. The proximal handle 112 connects to an intermediate handle 114.

In some embodiments, the intermediate handle 114 includes a longitudinal portion 116 that extends away from the proximal handle 112. Two transverse portions 118 extend oppositely and outwardly from the longitudinal portion 116. In some embodiments and as described in further detail below, the transverse portions 118 may be pulled to displace the intermediate handle 114 relative to the proximal handle 112 and deploy the anchoring device 102 from the insertion sheath 106.

The intermediate handle 114 may support a locking mechanism 120 that selectively locks and unlocks the intermediate handle 114 relative to the proximal handle 112. That is, the locking mechanism 120 selectively inhibits and permits movement of the intermediate handle 114 relative to the proximal handle 112. The locking mechanism 120 may generally include a user-pressable button 122, a user-pivotable dial or knob, or the like.

The insertion sheath 106 connects to the intermediate handle 114 opposite the proximal handle 112. In some embodiments, the insertion sheath 106 fixedly connects to the intermediate handle 114 so as to move with the intermediate handle 114 relative to the proximal handle 112. The insertion sheath 106 is formed by any of various materials appropriate for penetrating bone, such as metals (for example, stainless steel) and the like.

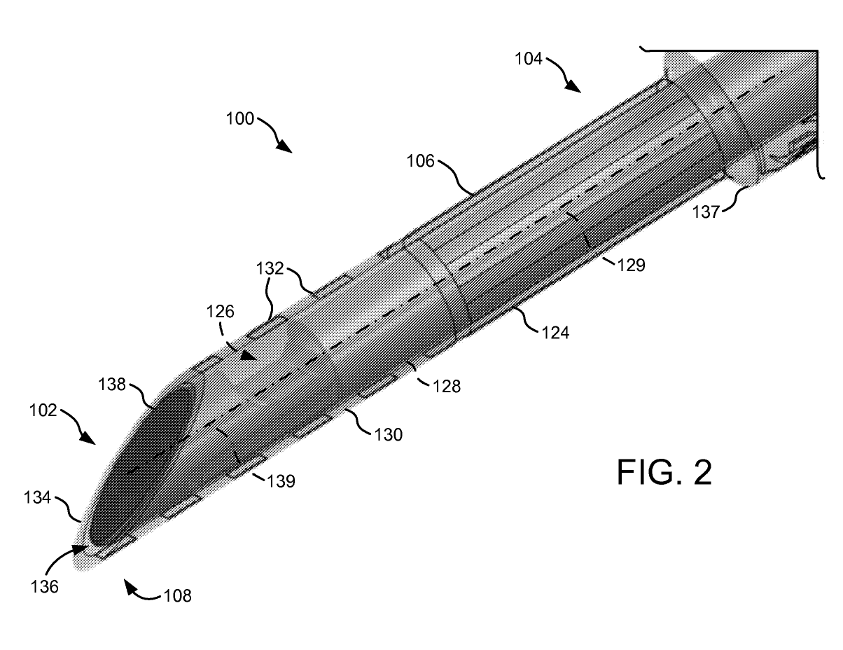

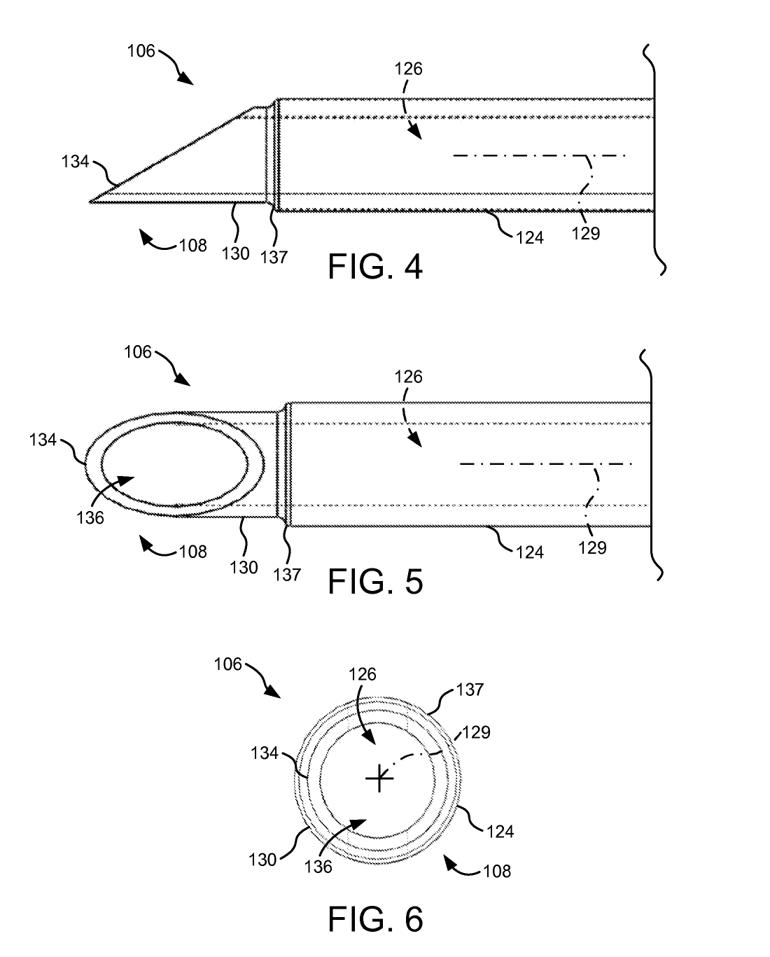

Referring to FIGS. 2-6, the insertion sheath 106 is generally a hollow, tube-shaped component. The insertion sheath 106 includes a sheath wall 124 that defines an internal or inner chamber 126. The inner chamber 126 detachably or releasably receives the anchoring device 102 at the distal end 108 of the insertion sheath 106.

In some embodiments, the inner chamber 126 also receives an ejection device 128 that is disposed proximate to and engages with the anchoring device 102. The ejection device 128 may generally be a cylinder-shaped component or a tube-shaped component. The ejection device 128 may be formed by various materials, such as metals, plastics, combinations thereof, and the like. The ejection device 128 may be movably received in the inner chamber 126 of the insertion sheath 106 and fixedly connected to the proximal handle 112. As such and as described in further detail below, the ejection device 128 moves with the proximal handle 112 relative to the intermediate handle 114 and the insertion sheath 106 to eject the anchoring device 102 from the insertion device 104 and into the bone of the subject.

The sheath wall 124 and the inner chamber 126 define a longitudinal axis 129 of the insertion device 104. That is, the longitudinal axis 129 of the insertion device 104 extends generally along the center of the insertion sheath 106.

Opposite the inner chamber 126, the insertion sheath 106 includes an outer surface 130. In some embodiments, the outer surface 130 includes one or more sets of longitudinally extending markings 132. The markings 132 facilitate determining the depth of penetration of the insertion sheath 106 and the anchoring device 102 into the bone of the subject. The markings 132 also facilitate determining the orientation of the sheath 106 and the anchoring device 102 in the bone of the subject.

The distal end 108 of the insertion sheath 106 penetrates the bone of the subject (in particular, first the cortical bone of the subject, and subsequently the cancellous bone of the subject) while the anchoring device 102 is carried by the insertion sheath 106 and inserted into the bone of the subject. Specifically, the distal end 108 includes a distal edge 134 that penetrates the bone of the subject while the anchoring device 102 is carried by the insertion sheath 106 and inserted into the bone of the subject. In some embodiments and depending on the thickness of the sheath wall 124, and as shown in the figures, the distal edge 134 may be considered a distal surface.

The distal edge or surface 134 defines a distal opening 136 of the insertion sheath 106. The distal opening 136 is coupled to the inner chamber 126 of the sheath 106. The anchoring device 102 also passes through the distal opening 136 to be deployed from the insertion device 104 and into the bone of the subject.

In some embodiments, the distal edge 134 and the distal opening 136 are angled relative to the longitudinal axis 129 of the insertion device 104 to facilitate penetrating the bone of the subject. Stated another way, the distal edge 134 and the distal opening 136 are disposed in a distal plane, and the distal plane is oriented at an obtuse angle relative to the longitudinal axis 129.

In some embodiments, the distal opening 136 has a shape, and the distal edge 134 is formed as the perimeter of the same shape to facilitate penetrating the bone of the subject. For example, in some embodiments, the distal opening 136 has a generally elliptical shape, and the distal edge 134 has the shape of the perimeter of an ellipse (that is, a hollow elliptical shape).

In some embodiments, the thickness of the sheath wall 124 may increase at a position between the distal end 108 and the handle 110. Such a construction may provide a depth stop 137 that engages the bone surface to limit the depth to which the insertion sheath 106 may penetrate into the bone of the subject.

Turning now to FIGS. 2, 7, and 8, the anchoring device 102 is detachably carried at the distal end 108 of the insertion sheath 106 (for example, the anchoring device 102 and the sheath wall 124 may be sized to provide a sliding fit between the components).

The anchoring device 102 engages the bone of the subject when the insertion sheath 106 penetrates the bone of the subject. The anchoring device 102 is reinforced by the insertion sheath 106 when the insertion sheath 106 penetrates the bone of the subject. As such, the anchoring device 102 may be formed by a variety of materials, including, for example, relatively soft materials. Relatively soft materials include materials preferred by some surgeons (for example, polyether ether ketone (PEEK)), bioabsorbable materials (for example, polymer-based bioabsorbable materials or magnesium-based bioabsorbable materials), and the like.

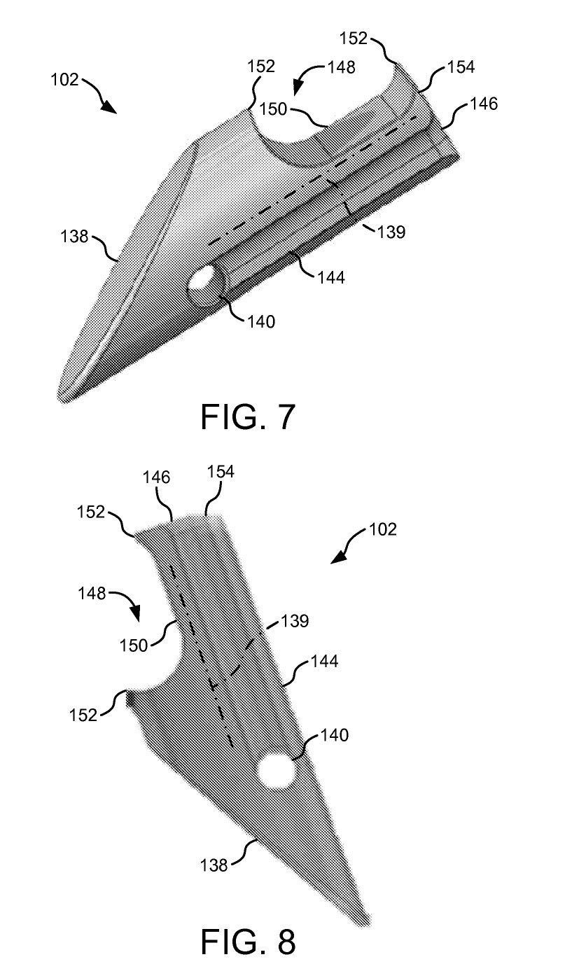

In some embodiments, the anchoring device 102 includes a longitudinal axis 139 (see FIGS. 7 and 8). Before the anchoring device 102 is deployed, the longitudinal axis 139 may be substantially collinear with the longitudinal axis 129 of the insertion device 104.

The anchoring device 102 includes a distal surface 138 that engages the bone of the subject when the insertion sheath 106 penetrates the bone of the subject. The distal surface 138 is disposed adjacent the distal opening 136 of the insertion sheath 106. Specifically, the distal surface 138 is substantially disposed in the distal plane defined by the distal end 108 of the insertion sheath 106 (that is, within +/−0.2 mm from being disposed in the distal plane). Similarly, the distal surface 138 of the anchoring device 102 may be oriented at substantially the same angle as the distal plane relative to the longitudinal axis 129 of the insertion device 104.

In some embodiments, the distal surface 138 of the anchoring device 102 substantially includes the same shape as the distal opening 136 of the insertion sheath 106 (that is, the distal surface 138 and the distal opening 136 have sufficiently similar shapes such that the anchoring device 102 is in contact or nearly in contact with the sheath wall 124 to reinforce the anchoring device 102 as described above). For example, in some embodiments, the distal surface 138 of the anchoring device 102 and the distal opening 136 of the insertion sheath 106 both have a generally elliptical shape.

In some embodiments, the longitudinal axis 139 of the anchoring device 102 substantially extends through the center of the distal surface 138.

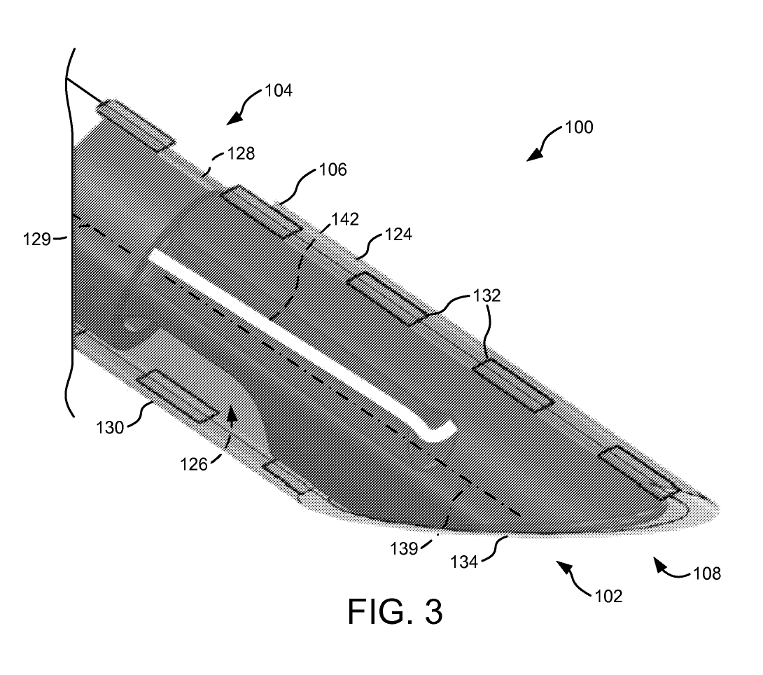

The anchoring device 102 also defines an eyelet 140 that receives a suture 142 (see FIG. 3). In some embodiments and as shown, for example, in FIG. 8, the eyelet 140 extends through anchoring device 102 in a direction that is substantially perpendicular to both the longitudinal axis 139 and the distal surface 138. In some embodiments, the eyelet 140 is offset from the longitudinal axis 139. With such a construction, after the anchoring device 102 is deployed from the insertion sheath 106, applying tension to the tails of the suture 142 causes the anchoring device 102 to pivot within the bone of the subject. Such an action, as described in further detail below, facilitates securing the anchoring device 102 to the bone of the subject.

In some embodiments, the anchoring device 102 includes channels 144 that extend proximally away from the eyelet 140. The channels 144 receive the tails of the suture 142, before the anchoring device 102 is deployed from the insertion sheath 106, to inhibit the suture 142 from being compressed between the anchoring device 102 and the sheath wall 124.

The anchoring device 102 also includes a proximal end 146 opposite the distal surface 138. In some embodiments, the proximal end 146 includes an engagement feature 148 that engages the bone of the subject after the anchoring device 102 is detached from the insertion sheath 106. For example and as shown in the figures, the engagement feature 148 may be provided by forming the anchoring device 102 with a recessed surface 150 and transversely extending teeth 152 disposed at the ends of the recessed surface 150. When pivoting the anchoring device 102 within the bone of the subject as described above, one or both of the teeth 152 may engage the bone of the subject to the secure the anchoring device 102 to the bone of the subject.

The proximal end 146 of the anchoring device 102 also includes a proximal surface 154. The proximal surface 154 may abut the ejection device 128 during bone penetration and deployment of the anchoring device 102 from the insertion device 104.

Referring briefly to FIG. 3, the suture 142 may be any of various sutures appropriate for coupling biological tissue. In addition and as described in further detail below, the suture 142 may be coupled to the anchoring device 102 in various manners. For example, the suture 142 may simply extend through the eyelet 140. As another example, the suture 142 may be coupled to the anchoring device 102 via a knot (not shown). Further still, the tails of the suture 142 may extend from the anchoring device 102, through the ejection device 128, through the handle 110, and into the slot of the proximal handle 112 to detachably secure the suture 142 to the insertion device 104. After the anchoring device 102 is deployed from the insertion device 104, the suture 142 may be coupled to the soft tissue of the subject in various manners, such as passing the tails of the suture 142 through the tissue or the like.

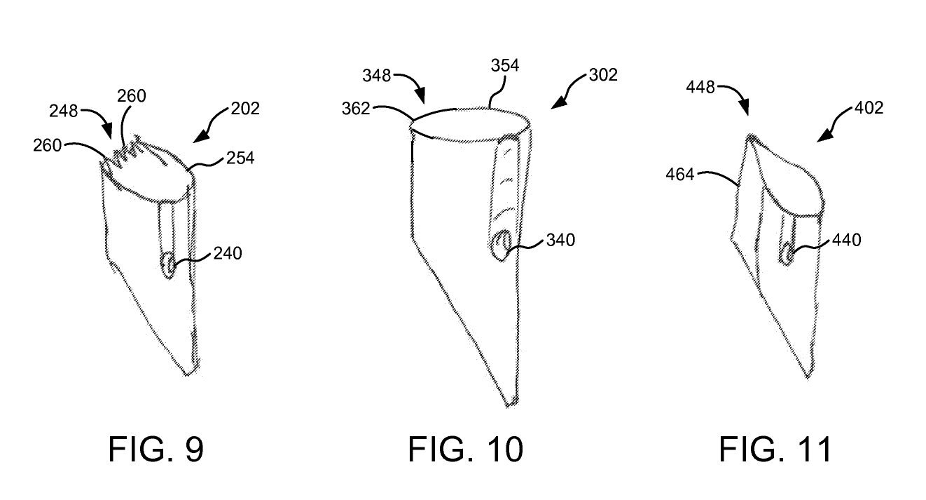

The suture anchor insertion apparatus may take other forms than those described above. For example, the anchoring device may include a different type of engagement feature. FIG. 9 illustrates an anchoring device 202 that is generally similar to the anchoring device 102 described above. However, the anchoring device 202 includes an engagement feature 248 that is formed as a set of barbs or teeth 260 disposed at the proximal surface 254 of the anchoring device 202. When pivoting the anchoring device 202 within the bone of the subject (for example, by applying tension to a suture, not shown, extending through the eyelet 240), one or more of the barbs 260 may engage the bone of the subject to the secure the anchoring device 202 to the bone of the subject.

FIG. 10 illustrates an anchoring device 302 that is generally similar to the anchoring device 102 described above. However, the anchoring device 302 includes an engagement feature 348 that is formed as the arcuate edge 362 of the proximal surface 354 of the anchoring device 302. When pivoting the anchoring device 302 within the bone of the subject (for example, by applying tension to a suture, not shown, extending through the eyelet 340), the arcuate edge 362 may engage the bone of the subject to the secure the anchoring device 302 to the bone of the subject.

FIG. 11 illustrates an anchoring device 402 that is generally similar to the anchoring device 102 described above. However, the anchoring device 402 includes an engagement feature 448 that is formed as a longitudinally extending edge 464 of the anchoring device 402. When pivoting the anchoring device 402 within the bone of the subject (for example, by applying tension to a suture, not shown, extending through the eyelet 440), the edge 464 may engage the bone of the subject to the secure the anchoring device 402 to the bone of the subject.

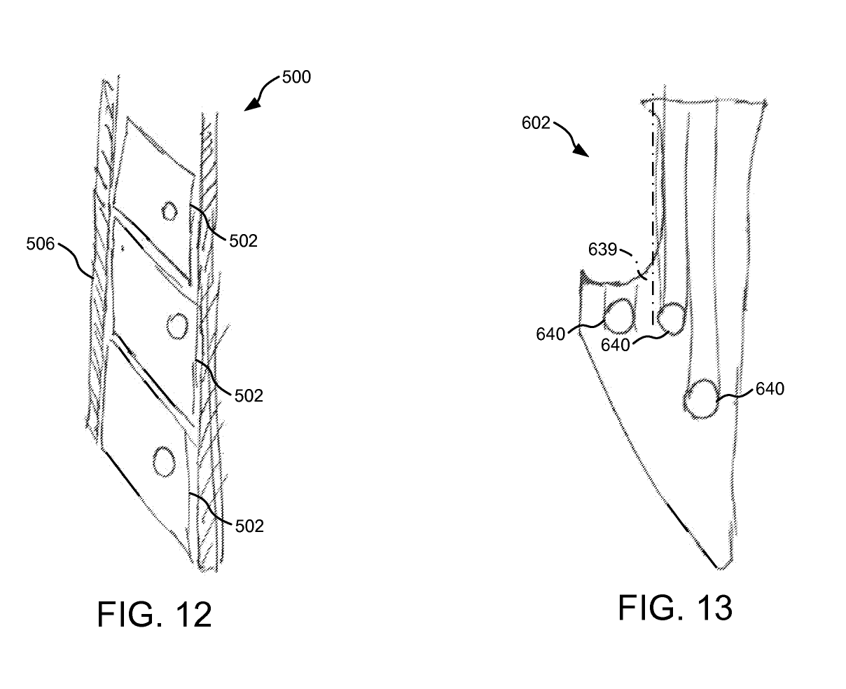

FIG. 12 illustrates a suture anchor insertion apparatus 500 in which the insertion sheath 506 detachably houses a plurality of anchoring devices 502. As shown in FIG. 12, the plurality of anchoring devices 502 includes three anchoring devices 502. In other embodiments, the plurality of anchoring devices 502 may include any other number of anchoring devices 502. The anchoring devices 502 may be substantially identical (that is, the devices 502 may have the same dimensions within manufacturing tolerances) and may include the features of any of the anchoring devices described herein. The anchoring devices 502 may be sequentially deployed from the insertion sheath 506.

FIG. 13 illustrates an anchoring device 602 that is generally similar to the anchoring device 102 described above. However, the anchoring device 602 includes a plurality of eyelets 640. As shown in FIG. 13, the plurality of eyelets 640 includes three eyelets 640. In other embodiments, the plurality of eyelets 640 may include any other number of eyelets 640. In some embodiments, each of the eyelets 640 is offset from the longitudinal axis 639 of the anchoring device 602. Each of the eyelets 640 may be coupled to a different suture (not shown) that identifies the position of the associated eyelet 640 (for example, via color coding, detachable labels, or the like). From this information, a user may determine how the anchoring device 602 will pivot within the bone of the subject if tension is applied to one of the sutures.

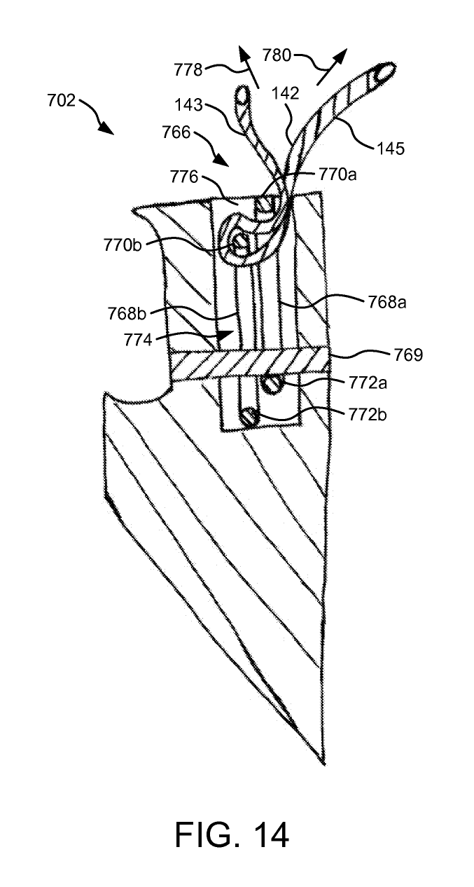

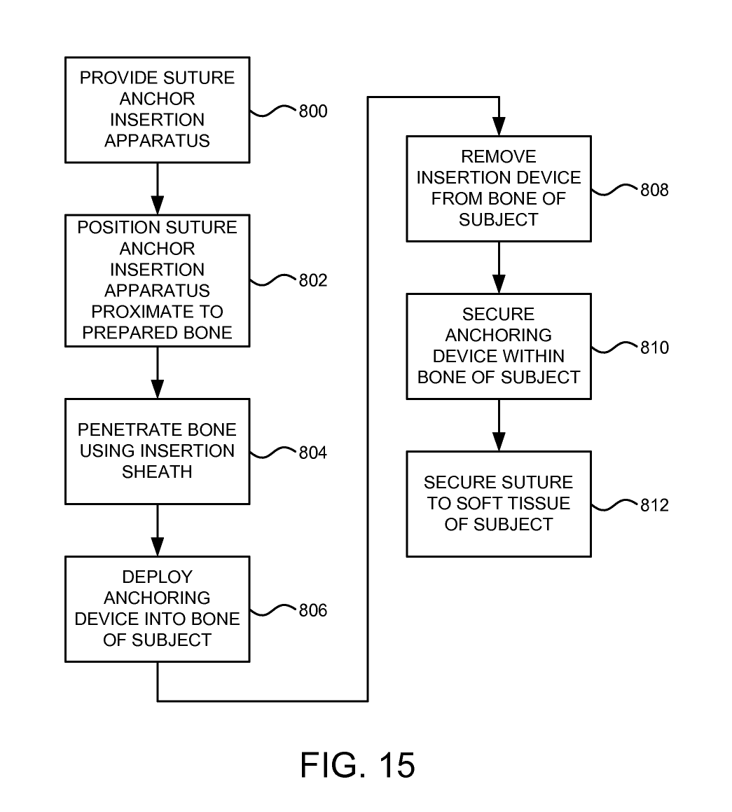

FIG. 14 illustrates an anchoring device 702 that is generally similar to the anchoring device 102 described above. However, the anchoring device 702 also includes a suture cinching or locking assembly 766 such as those described in U.S. Pat. No. 7,938,847, entitled “RING CINCH ASSEMBLY TO ATTACH BONE TO TISSUE”, which is hereby incorporated by reference in its entirety. For example, the cinching or locking assembly 766 may be similar to the locking assembly shown in FIGS. 30A-30D and described in the associated portions of the detailed description of U.S. Pat. No. 7,938,847.

In some embodiments, the locking assembly 766 includes a plurality of locking elements. The locking elements may be locking rings 768a and 768b that are secured by a transversely-extending pin or cross bar 769. The locking rings 768a and 768b include upper bars 770a and 770b and lower bars 772a and 772b, respectively. The locking ring 768a is fixed to the pin 769, and the locking ring 768b is sized to be longitudinally movable within a cavity 774 defined within the anchoring device 702.

In some embodiments, the suture 142 is threaded through the locking elements 768a and 768b in the manner shown in FIG. 14. Applying tension to a first portion 143 of the suture 142, which may be the tails of the suture 142, in the direction indicated by arrow 778 causes the suture 142 to move through the locking assembly 766 in direction 778. An opposite second portion 145 of the suture 142 may be a loop coupled to soft tissue of the subject. Thus, applying tension to the first portion 143 of the suture 142 in direction 778 draws the soft tissue toward the anchoring device 702.

The locking assembly 766 also inhibits movement or slippage of the suture 142 and the soft tissue in an opposite direction. That is, applying tension to the second portion 145 of the suture 142 in the direction indicated by arrow 780 causes the suture 142 to apply an upward force to the upper bar 770b. This causes the upper bars 770b and 770a to interact such that the suture length positioned in a suture gap 776 is compressed between the upper bars 770a and 770b, thereby inhibiting movement of the suture 142 and the soft tissue in direction 780.

FIGS. 15-20 illustrate an exemplary method for coupling soft tissue of a subject to bone of the subject according to embodiments of the present invention. The method may use, for example, the suture anchor insertion apparatus 100 described above. The method described below includes the reference numbers associated with the apparatus 100, although it is to be understood that the method could be performed with other suture anchor insertion devices (for example, those including the anchoring devices 202, 302, 402, 502, 602, or 702).

At block 800, the method begins by providing a suture anchor insertion apparatus 100 in which the anchoring device 102 is loaded into the insertion sheath 106. The suture anchor insertion apparatus 100 may also be provided with the tails of the suture 142 extending through the insertion device 104 and secured within the slots of the proximal handle 112. In some embodiments, the loaded suture anchor insertion apparatus 100 may be provided in, and subsequently removed from, sterilized packaging.

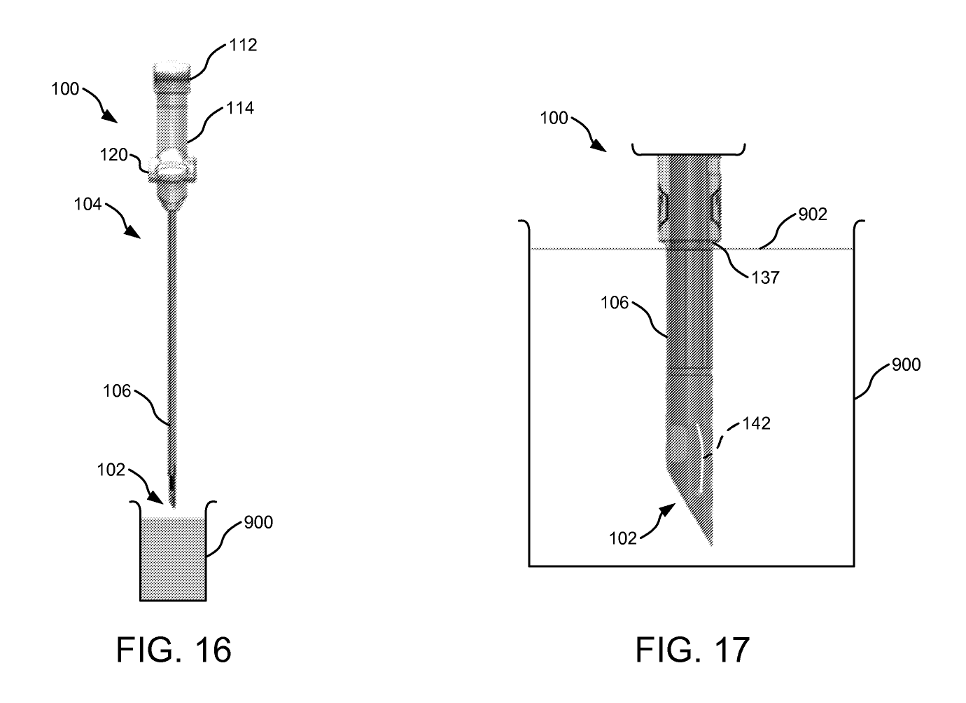

At block 802, a user (for example, a surgeon) positions the suture anchor insertion apparatus 100 proximate to prepared bone 900 of a subject (see FIG. 16).

At block 804, the insertion sheath 106 penetrates the bone 900 of the subject and the anchoring device 102 engages the bone 900 of the subject (see FIG. 17). In some embodiments, the proximal handle 112 may be impacted or struck by a tool, such as a mallet (not shown), to cause the insertion sheath 106 to penetrate the bone 900 of the subject. The insertion sheath 106 penetrates the bone 900 until the anchoring device 102 is disposed at a desired depth. In some embodiments, the anchoring device 102 is disposed at the desired depth when the depth stop 137 engages the surface 902 of the bone 900.

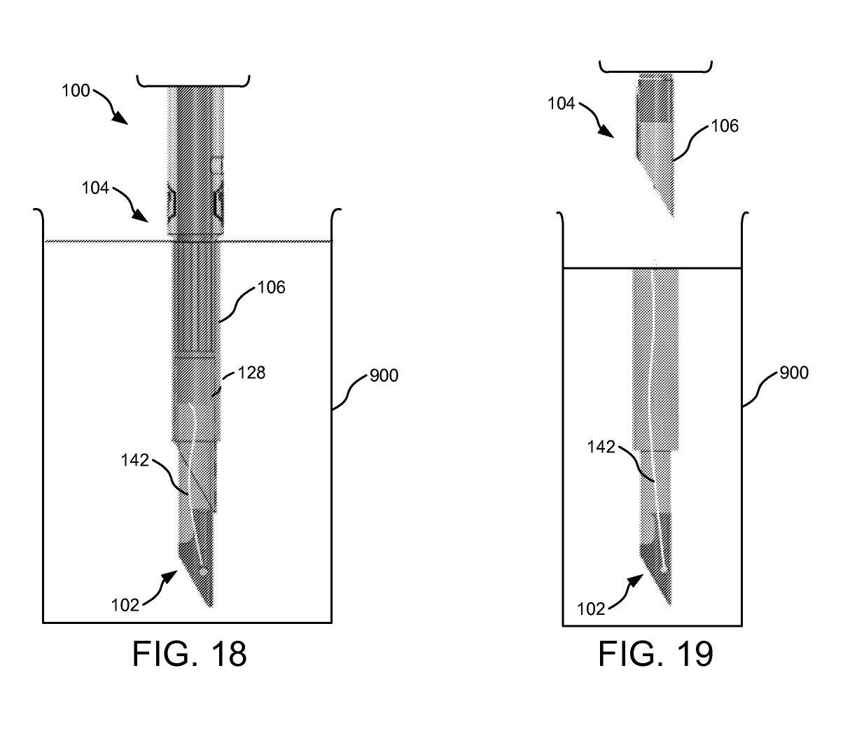

At block 806, the anchoring device 102 is deployed from the insertion device 104 and into the bone 900 of the subject (see FIG. 18). In some embodiments, this is achieved by first removing the tails of the suture 142 from the slots of the proximal handle 112, disengaging the locking mechanism 120 (for example, the user-pressable button), and then translating the intermediate handle 114 relative to the proximal handle 112. This causes the insertion sheath 106 to retract relative to the ejection device 128 and deploy the anchoring device 102 from the insertion device 104 and into the bone 900 of the subject.

In some embodiments, the proximal handle 112 may also be impacted or struck by a tool, such as a mallet, to cause the insertion sheath 106 to penetrate the bone 900 of the subject after disengaging the locking mechanism 120 and before deploying the anchoring device 102 from the insertion device 104.

At block 808, the insertion device 104 is removed from the bone 900 of the subject (see FIG. 19). The insertion device 104 is then discarded.

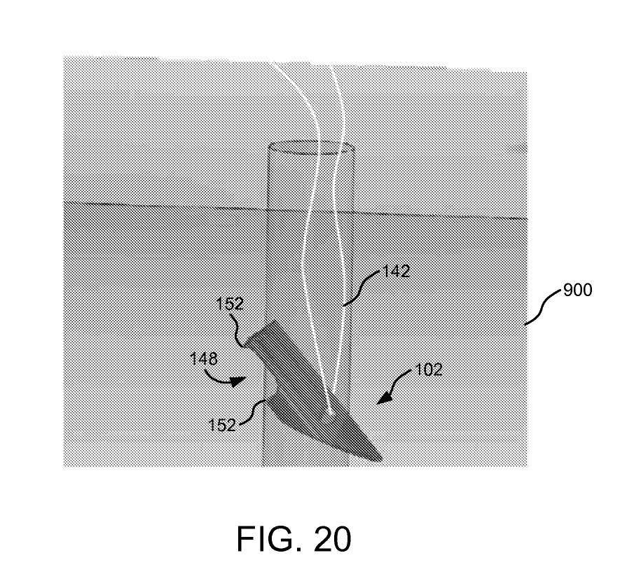

At block 810, the anchoring device 102 is secured within the bone 900 of the subject. In some embodiments, this is achieved by applying tension to the tails of the suture 142 to pivot the anchoring device 102 within the bone 900 of the subject (see FIG. 20). The engagement feature 148 (for example, one or both of the teeth 152) may engage the bone 900 of the subject to the secure the anchoring device 102 to the bone 900 of the subject.

At block 812, the tails of the suture 142 are passed through the soft tissue of the subject to secure the soft tissue to the bone 900 of the subject.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A suture anchor insertion apparatus comprising:
an insertion device including an insertion sheath, the insertion sheath including an inner chamber and a distal end, the distal end being adapted to penetrate bone of a subject, and the distal end defining an opening in communication with the inner chamber;
an anchoring device carried by the insertion sheath within the inner chamber, the anchoring device including a distal surface adapted to engage the bone of the subject when the distal end of the insertion sheath penetrates the bone of the subject, and the anchoring device being adapted to pass through the opening to detach from the insertion sheath and couple to the bone of the subject; and
a suture coupled to the anchoring device and being adapted to couple to soft tissue of the subject.

2. The suture anchor insertion apparatus of claim 1, wherein the distal surface of the anchoring device includes a shape, and the opening of the insertion sheath substantially includes the shape of the distal surface.

3. The suture anchor insertion apparatus of claim 1, wherein the insertion sheath defines a longitudinal axis of the insertion device, the distal end of the insertion sheath includes a distal edge defining the opening, the distal edge and the opening being disposed in a distal plane, the distal plane being disposed at an obtuse angle relative to the longitudinal axis, and wherein the distal surface of the anchoring device is substantially disposed in the distal plane.

4. The suture anchor insertion apparatus of claim 3, wherein the distal surface of the anchoring device includes a shape, and the opening of the insertion sheath substantially includes the shape of the distal surface.

5. The suture anchor insertion apparatus of claim 4, wherein the shape is an elliptical shape.

6. The suture anchor insertion apparatus of claim 1, wherein the anchoring device includes a proximal end opposite the distal surface, and the proximal end includes an engagement feature adapted to engage the bone of the subject after the anchoring device is detached from the insertion sheath.

7. The suture anchor insertion apparatus of claim 1, wherein the anchoring device includes a longitudinal axis and an eyelet configured to receive the suture, and the eyelet is offset from the longitudinal axis such that, after the anchoring device is detached from the insertion sheath, applying tension to the suture causes the anchoring device to pivot within the bone of the subject.

8. The suture anchor insertion apparatus of claim 7, wherein the anchoring device includes a proximal end opposite the distal surface, and the proximal end includes an engagement feature adapted to engage the bone of the subject when the anchoring device pivots within the bone of the subject.

9. A suture anchor insertion apparatus comprising:
an insertion device including an insertion sheath, the insertion sheath defining a longitudinal axis of the insertion device, and the insertion sheath including an inner chamber and a distal end, the distal end being adapted to penetrate bone of a subject, and the distal end including:
a distal edge disposed in a distal plane, the distal plane being disposed at an obtuse angle relative to the longitudinal axis;
an opening defined by the distal edge and disposed in the distal plane, the opening being in communication with the inner chamber;
an anchoring device carried by the insertion sheath within the inner chamber, the anchoring device including a distal surface substantially disposed in the distal plane, and the anchoring device being adapted to pass through the opening to detach from the insertion sheath and couple to the bone of the subject; and
a suture coupled to the anchoring device and being adapted to couple to soft tissue of the subject.

10. The suture anchor insertion apparatus of claim 9, wherein the distal surface of the anchoring device includes a shape, and the opening of the insertion sheath substantially includes the shape of the distal surface.

11. The suture anchor insertion apparatus of claim 10, wherein the shape is an elliptical shape.

12. The suture anchor insertion apparatus of claim 9, wherein the insertion sheath comprises a first material having a first hardness, the anchoring device comprises a second material having a second hardness, and the second hardness is less than the first hardness.

13. The suture anchor insertion apparatus of claim 12, wherein the second material is a bioabsorbable material.

14. The suture anchor insertion apparatus of claim 12, wherein the second material is polyether ether ketone.

15. The suture anchor insertion apparatus of claim 12, wherein the distal surface of the anchoring device is adapted to engage the bone of the subject when the distal end of the insertion sheath penetrates the bone of the subject, and the distal end of the insertion sheath is adapted to reinforce the anchoring device when the distal end of the insertion sheath penetrates the bone of the subject.

16. A suture anchor insertion apparatus comprising:
an insertion device including an insertion sheath, the insertion sheath including an inner chamber and a distal end, the distal end being adapted to penetrate bone of a subject, the distal end defining an opening in communication with the inner chamber, and the opening having a shape;
an anchoring device carried by the insertion sheath within the inner chamber, the anchoring device including a distal surface, the distal surface being disposed adjacent the opening and substantially including the shape of the opening, and the anchoring device being adapted to pass through the opening to detach from the insertion sheath and couple to the bone of the subject; and
a suture coupled to the anchoring device and being adapted to couple to soft tissue of the subject.

17. The suture anchor insertion apparatus of claim 16, wherein the insertion sheath comprises a first material having a first hardness, the anchoring device comprises a second material having a second hardness, and the second hardness is less than the first hardness.
